(19) United States
(12) Patent Application Publication
(10) Pub. No.: US 2018/0042759 A1
(43) Pub. Date: Feb. 15, 2018

(54) PRE-PROGRAMMED ANTERIOR GUIDANCE PACKAGE, KIT, AND METHOD

(71) Applicant: Kelly LUCAS, Wasilla, AK (US)

(72) Inventor: Kelly LUCAS, Wasilla, AK (US)

(21) Appl. No.: 15/791,830

(22) Filed: Oct. 24, 2017

Related U.S. Application Data
(63) Continuation of application No. 13/573,283, filed on Sep. 6, 2012.

(51) Int. Cl. A61F 5/56; A61C 7/36

(52) U.S. Cl. CPC A61F 5/566; A61C 7/36

(57) ABSTRACT

A pre-fabricated, pre-programmed or custom-made Anterior Guidance Package (AGP) including a maxillary guidance component and a mandibular guidance component configured to attach to respective maxillary and mandibular retention pieces, wherein the maxillary guidance component and mandibular guidance component are configured to function against each other to provide anterior guidance to a mandible of a user.

### CROSS-REFERENCE TO RELATED APPLICATIONS

This application is a continuation application of U.S. patent application Ser. No. 13/573,283 filed Sep. 6, 2012, the content of which is incorporated by reference herein in its entirety.

### FIELD OF THE INVENTION

Current application relates to an anterior guidance package, especially an anterior guidance package pre-fabricated in various sizes and shapes to be used to produce a superior night guard for the amelioration of the damage and pain caused by bruxism.

### BACKGROUND OF THE INVENTION

Bruxism is an inappropriate activity that causes many dental and medical problems. Dental conditions to include malocclusion and centric relation/centric occlusion discrepancy can amplify the damage caused by bruxism. Some of the problems include myo-facial pain syndrome, damage to teeth, and damage to the temporo-mandibular joints (TMJ). Many kinds of ‘night guards’ have been developed to ameliorate the negative impacts of bruxism. These include simple coverage of teeth, appliances that correct the centric relation/centric occlusion discrepancy which allow the TMJ to relax in its most anatomically appropriate and best stress bearing position (centric relation), and the provision of anterior guidance, which among other benefits reduces significantly the inappropriate muscle force associated with bruxism.

The best night guards are those that combine all three of these features. Simple coverage of teeth does help reduce damage to teeth by providing a barrier. However, without centric occlusion/centric relation (CO/CR) discrepancy correction and without anterior guidance, this type of night guard could actually cause increased severity of bruxism. As a result, causes worse myo-facial pain syndrome and a greater tendency toward TMJ damage.

The correction of a centric occlusion/centric (CO/CR) relation discrepancy eliminates deviating tooth contacts in the posterior occlusion allowing the condyles to seat into their most comfortable positions. By eliminating the contact of deviating inclines of teeth in a malocclusion there will be no proprioceptive message to muscles to deviate around that interference. When the muscle stops being stimulated into holding the mandible in a deviated position, then normal muscle activity can resume and spasticity will cease. Anterior guidance refers to particular function of anterior teeth to provide physical limits of movement of the front end of the mandible. Appropriate anterior guidance in centric relation position, long centric, straight protrusive and lateral excursions protects back teeth and reduces the muscle forces of bruxism because of the mechanical advantageous position of being anterior to the muscle power used to close the mandible.

Traditionally, dentists have been able to improve CO/CR discrepancies and anterior guidance by creating a custom made and custom adjusted night guard, which typically are attached to the maxillary or mandibular teeth and opposed by natural teeth. This appliance is custom built for a patient by a dentist considering their particular malocclusion and other factors allowing the mandible to be in centric relation with appropriate anterior guidance giving the patient significant relief from the damage and pain of bruxism. However, a dentist must spend a lot of time and effort to custom create and custom modify a night guard to try to achieve appropriate anterior guidance and reduce CO/CR discrepancy for a patient's particular malocclusion. These efforts are further complicated by missing teeth or periodontally weakened teeth. The patient must also spend a lot of time to achieve the desired result and a high cost. It is the purpose of the current invention to provide a means to allow a dentist or even a non-dentist to create a superior night guard that is easier and faster to make and more affordable for the patient. It is another purpose of the current invention to remove the variable factors of maloccluded teeth, missing or periodontally weakened teeth. The third purpose of the current invention is to provide superior anterior guidance and simultaneously obliterate any CO/CR discrepancy by an individual who does not necessarily possess the specialized knowledge of a dentist.

### DESCRIPTION OF THE RELATED ARTS

U.S. Patent Application Publications 2010/0279246, 2007/0178420, 2007/0099144 by Keski-Nisula, Katri et al. disclose an odontological device and device series to guide an individual's occlusion and a method to be used in selecting an occlusion guidance appliance device to be used in orthodontic treatment. This kind of device contains a U-shaped arch with a lower surface on the side of the lower jaw and a higher surface on the side of the upper jaw, and in both of which there are concaves in which to place the individual's teeth, and where the bottoms of the concaves form of the isthmus separating the concaves from one another.

U.S. Patent Application Publication 2008/0000483, U.S. Pat. Nos. 6,161,542, 6,041,784, and 5,365,945 to Halstrom disclosed an intra-oral dental appliance for treatment of sleep disorders including snoring, sleep apnea and nocturnal bruxism. The appliance includes an upper member conforming to the patient's maxillary dentition; a lower member conforming to the patient's mandibular dentition; and a connecting assembly for adjustable coupling the upper and lower members together. The only benefit in regard to bruxism is that Halstrom's appliance does separate teeth therefore damage to teeth would be eliminated. However, his connecting assembly places the jaw in an unnatural position. This may cause many problems because the major goal of treatment for tooth damage, myofacial pain, migraines etc secondary to bruxism, is to allow the jaw (mandible) freedom to relax to its most comfortable position. This position would be centric relation for 99% of people. Centric relation allows the jaw to be in its most anatomically correct stress bearing position and the place where the muscles are most calm. Dentists use centric relation or an even more refined point to create a night guard that allows the mandible to rest there and then guidance from that position to avoid posterior interferences and freedom so the jaw can move, the patient can yawn, open, sneeze, breathe, swallow etc. normally.

When a person, having malocclusion, closes their mouth, the jaw is forced to adapt a position other than centric relation. Because of muscle engrams the jaw ends up living in this inappropriate position. By locking the lower jaw forward in relation to the upper jaw over time, this will happen when a person wears Halstrom's appliance, the person may experience unintended and inappropriate orthodontic movement of the teeth that create or make worse the malocclusion. By locking the lower jaw forward in relation to the upper jaw you have pulled the mandibular condyle down the articular imminence to a very inappropriate position (not in the fossa). It may prevent damage to teeth but if the person exerts muscle activity in that position one is more likely to damage the TMJ. One major purpose of a night guard is to allow the persons jaw to assume the position of centric relation, not purposely pull the jaw into some other position.

Myofacial pain would be terrible for a person wearing this type appliance since the condyles and muscles of mastication are artificially pulled into very inappropriate positions.

U.S. Patent Application Publication 2005/0288624, U.S. Pat. Nos. 7,654,267, 5,795,150 (which is assigned to NTU-tts, Inc.), and U.S. Pat. No. 5,085,584 by Boyd, and U.S. Pat. No. 6,666,212 to Boyd. Sr., illustrate an intraoral discluder for preventing chronic tension headaches, common migraine headaches, and temporo-mandibular disorders that are caused or perpetuated by chronic activity of the temporalis muscle. The discluder includes a trough, contoured to encompass at least one maxillary or mandibular incisor, from which extends a protruding platform, for engagement by the opposing incisors. The trough can be retained on the teeth by any adaptable material than can flow around the teeth and then maintain its shape. Once in place in the wearer's mouth, one or two opposing incisors will come into contact with the platform prior to the upper and lower posterior and/or canine teeth coming into contact, regardless of the position of the mandible, thereby reducing the intensity of the activity of the temporalis muscle. In addition, a special post on the discluder's platform is engageable directly with one or more opposing incisors, to act as a stop and thereby inhibit excessive retrusive movement of the mandible and urge the mandible toward a more protrusive position. This can reduce the intensity of undesired clenching, and it can enhance the size of the wearer's pharyngeal airspace, thereby reducing the incidence and severity of snoring.

However, Boyd's invention did not consider patients who have malocclusion, loss of teeth and weak teeth, etc. If a patient with such abnormalities on teeth wears Boyd's intraoral discluder and brux while sleep and functions against these teeth, it could make the patient's tooth problems worse. Also, many people have reported chipping teeth which oppose this appliance. Also, because the guidance is flat there are many malocclusions that to disclude the mandible enough to avoid the posterior interferences the vertical dimension of the appliance could become so big as to make the appliance uncomfortable or impossible to wear. This appliance therefore cannot intervene as effectively to as many types and severities of malocclusions as the current invention.

On the web site of http://www.nti-tss.com NTI-tts, Inc. commercially broadcast a new intraoral discluder for preventing chronic tension headaches, common migraine headaches, and temporo-mandibular disorders that are caused or perpetuated by chronic activity of the temporalis muscle. That product is shown in the U.S. Pat. No. 6,666,212 to Boyd Sr. But, NTI-tts modified the Boyd's invention slightly by developing a three-dimensional guidance on the surface by trial and error practice of a dentist. They said it usually take couple of hours to finish that “opposing slider.”

In that video, a dentist engages a preliminary “opposing slider” on mandibular incisors of a patient and ask the patient whether the “pain” is gone or better. If the patient says ‘no’, the dentist takes it out of the patient's mandibular incisors and cut the surface again and again until the patient says ‘better.’ So trial and error customization by a dentist is still necessary, and is not so different from the traditional method of carving anterior guidance under the lower surface of maxillary retention piece of old splint. The only difference is that the “opposing slider” is smaller and cheaper. It does not eliminate the trial and error method performed by a dentist. The material may be cheaper than old splint. But there is still a specific labor cost of the dentist that must be done. And the patient must wait until the dentist carves a right shape for the patient. Also, because the anterior guidance that is created is flat therefore the vertical dimension of the entire appliance will be larger even when the patients' mandible is at rest, not necessarily just in an excursion. This increase in vertical dimension could be so excessive as to preclude many patients from being able to utilize NTI-tss.

U.S. Pat. No. 4,773,854 to Weber disclosed herein is a device for the representation of condylar movements of a patient and their correct simulation which includes models of sets of teeth to determine the required corrections to the biting surfaces in order to obtain ideal occlusion. The device includes an articulator with the lower part thereof able to be brought into a predetermined three-dimensional relation with respect to an upper part of the articulator and having two blocks having guide elements on the lower part of the articulator to support condyle balls of the upper part of the articulator. The device further includes a lower jaw recording bow and an upper jaw recording bow which can be brought into an active and predetermined relation with respect to the articulator and which disposes of at least three recording plates with corresponding recording pins as well as positioning spoons for the combination of a lower jaw dentition model. With this device, opening movements of articulation may be recorded three-dimensionally so that three clear crossing points are created for the occlusion.

U.S. Pat. No. 4,901,737 to Toone discloses an intra-oral appliance for reducing snoring which repositions the mandible in an inferior (open) and anterior (protrusive) position as compared to the normally closed position of the jaw. Once the dentist or physician determines the operative “snore reduction position” for a particular patient, an appropriate mold is taken of the maxillary dentition and of the mandibular dentition for formation of the appliance template. The Toone appliance includes a pair of V-shaped spacer members formed from dental acrylic which extend between the maxillary and mandibular dentition to form a unitary mouthpiece. In an alternative embodiment of the Toone invention, the spacer members are formed in two pieces and a threaded rod is provided to enable adjustment of the degree of mandibular protrusion or retrusion after the mouthpiece is formed.

European patent application No. 0,312,368 published also discloses an intra-oral device for preventing snoring. This device consists of a U-shaped mouthpiece which conforms to the upper dental arch of the user and includes a sloped, lower ramp for engaging the mandibular dentition. Normal mouth motions, such as the clenching of the jaw, will cause some of the mandibular dentition to engage the underside of the ramp, thereby camming the lower jaw forward to increase the spacing between the base of the tongue and the posterior wall of the pharynx.

U.S. Pat. No. 5,722,828 to Halstrom discloses an apparatus and method for producing a gothic arch tracing representative of the natural range of motion of a patient's mandible. The apparatus consists of a kit including a mandibular bite rim having a tracing plate; a maxillary bite rim having a tracing arm; and a stylus releasably connectable to the tracing arm for extending between the tracing arm and the tracing plate externally of the patient's mouth. The stylus has a marker on one end thereof for drawing a gothic arch tracing on a removable paper substrate, such as a post-it note, attachable to the tracing plate. The tracing is used in the fabrication of a dental bite registration mold for the patient. The mold may in turn be used to mount casts of the patient's dentition in a specific relationship as required for prosthetic or therapeutic purposes.

Frank et al, disclosed a full contact splint with anterior guidance on the internet at address http://www.greatlakesortho.com/content/files/resources/SplintApplianceSelectionGuide_S222.pdf. The full contact splint with anterior guidance is to form an anterior guidance under the lower surface of the maxillary retentive piece. However, developing anterior guidance directly to the lower surface of the maxillary retentive piece is very time-consuming trial and error job and expensive for both of dentist and patient.

From the above prior art it is found that none of the prior art provides the ability to treat such a broad range of malocclusions in the context of bruxism or as economical, easy to apply, and medically safe anterior guidance package for patients with various malocclusion as provided in the current application.

### SUMMARY

Many people who inappropriately brux or clench also have the complicating factors of a malocclusion that caused discrepancy between centric occlusion and centric relation. Another factor that can amplify the pain and damage potential of bruxing/clenching is inadequate anterior guidance. Bruxism and bruxism combined with these factors can cause myofacial pain syndrome and many other types of damage to the teeth and TMJ (Temporo-mandibular Joint). Many kinds of ‘Night Guards’ have been provided to allow the mandibular condyles to locate in their most comfortable position by freeing mandible from malocclusions and posterior interferences. Anterior guidance is a physical limitation of all excursions of the jaw. Elimination of centric relation/centric occlusion discrepancies allows the patient to be free of their malocclusion to allow the patients jaw to acquire centric relation position. Anterior guidance and freedom of the jaw from centric relation/centric occlusion discrepancies can be provided in a custom fabricated and custom adjusted acrylic night guard made by a dentist on the incisal surface of the maxillary teeth or mandibular teeth. However, a dentist must spend lot of time and effort to create and modify a customized night guard for the patient to create anterior guidance and relief from centric occlusion/centric relation discrepancy in consideration of their particular malocclusion. It also burdens the patient with time and money. It is purpose of the current invention to provide an anterior guidance package for a splint, superior anterior guidance installed night guard, which is more affordable for patients and easier to create for a dentist, even for non-dentist. Another purpose of the current invention is to provide an anterior guidance package for an easy to make anterior guidance installed night guard for patients of various malocclusions. A pre-fabricated, pre-programmed or custom-made guidance assembly Anterior Guidance Package (AGP) is provided. The anterior guidance package of the current invention is comprised of one maxillary guidance component and one mandibular guidance component. Those guidance components are attached to the maxillary retention piece and mandibular retention piece of a splint, respectively, to provide superior anterior guidance to the mandible. An AGP splint kit according to current application can provide a fast, inexpensive, easy way to construct a high quality anterior guidance equipped night guard (orthotic appliance) that will be superior to a custom appliance constructed by a dentist.

### DETAILED DESCRIPTION

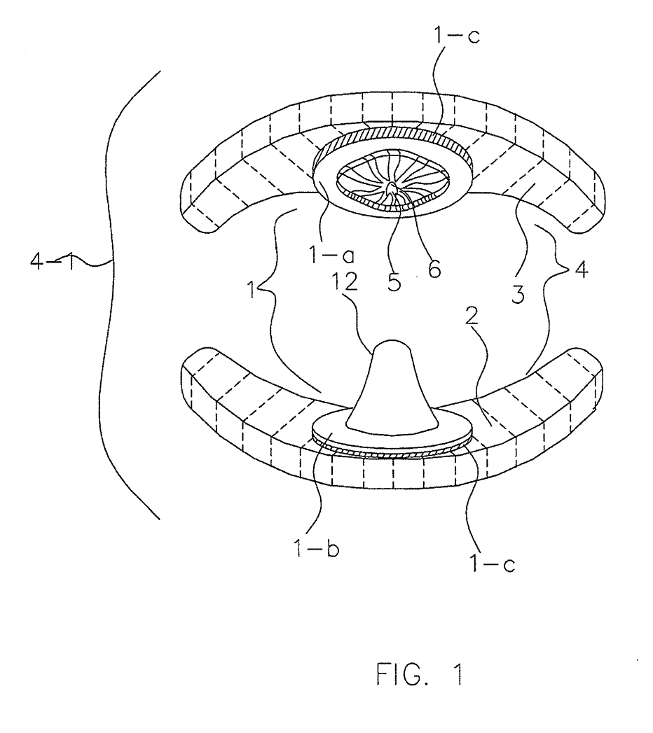

FIG. 1 is an exploded view of the Anterior Guidance Package (AGP) according to current invention connected to a mandibular retention piece and a maxillary retention piece of a splint.

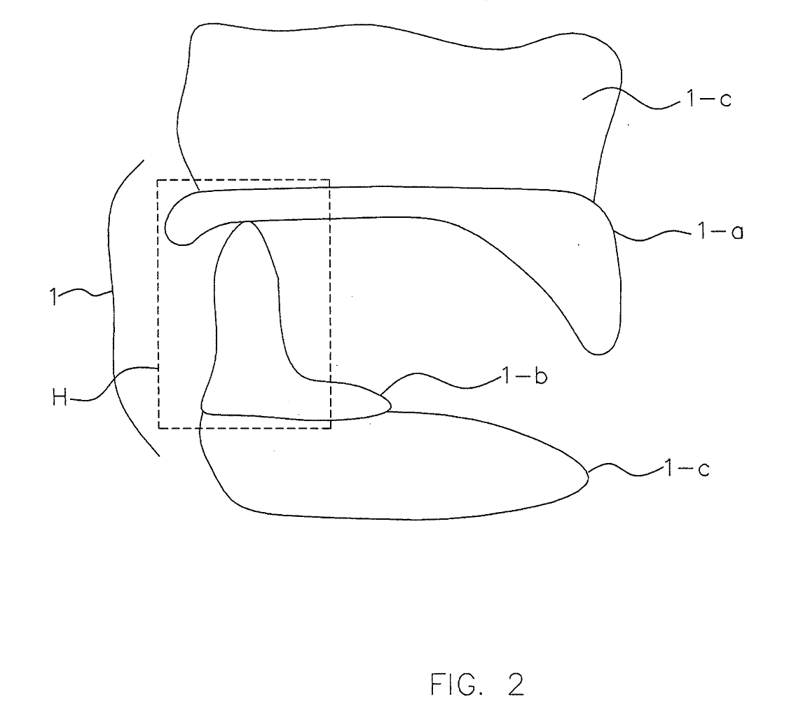

FIG. 2 is a cross-sectional side view of the Anterior Guidance Package (AGP) assembled in its correct orientation by a removable holder before it is indexed onto the maxillary and mandibular retentive pieces.

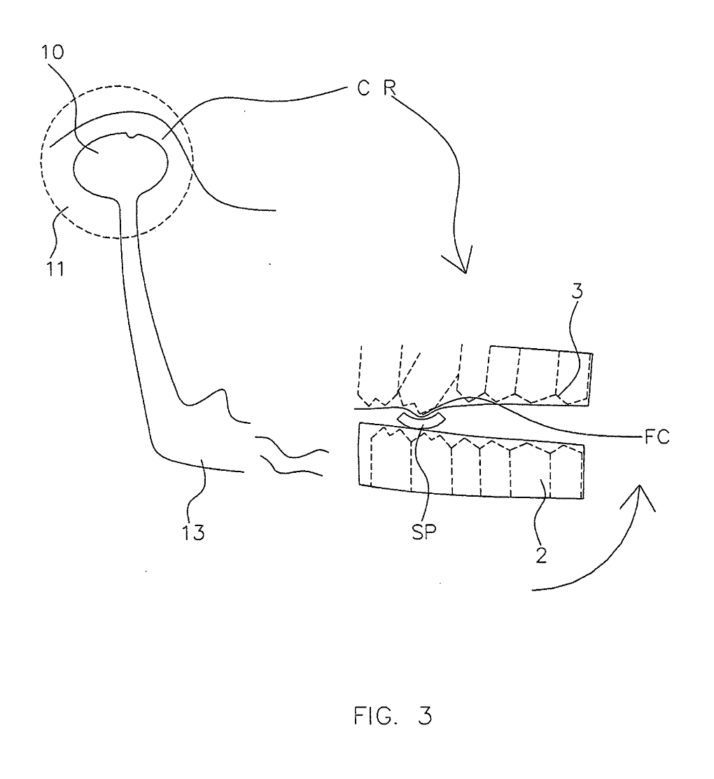

FIG. 3 is a perspective cross-sectional view of the mandible hinging up in centric relation finding the first contact on teeth or retentive pieces and placing a spacer at that location.

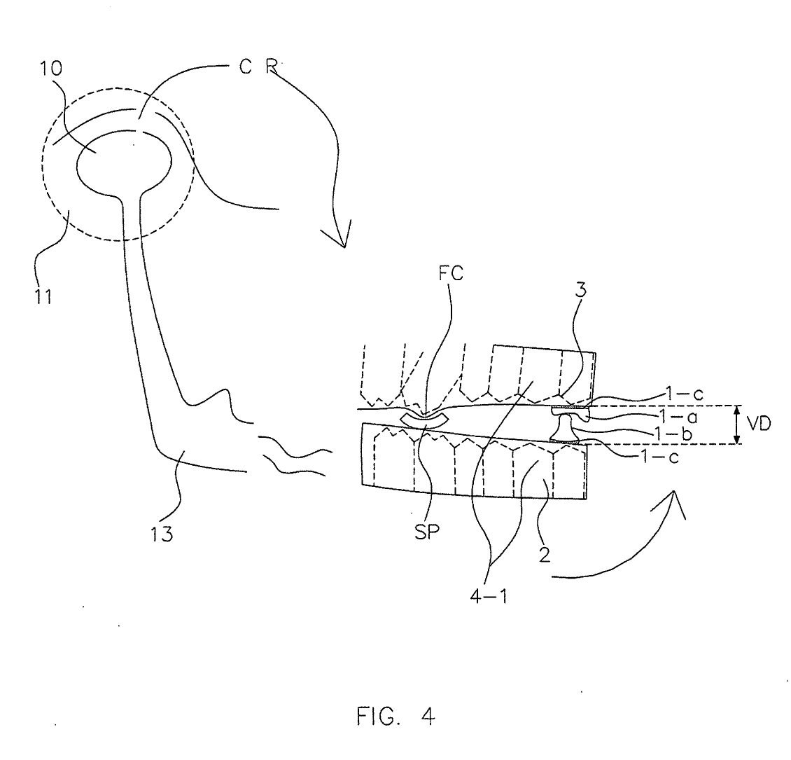

FIG. 4 is a perspective cross-sectional view of the mandible hinging up in centric relation indexing the Anterior Guidance Package (AGP) onto the retentive pieces when the mandible is in centric relation and the vertical dimension is appropriate according to the first contact.

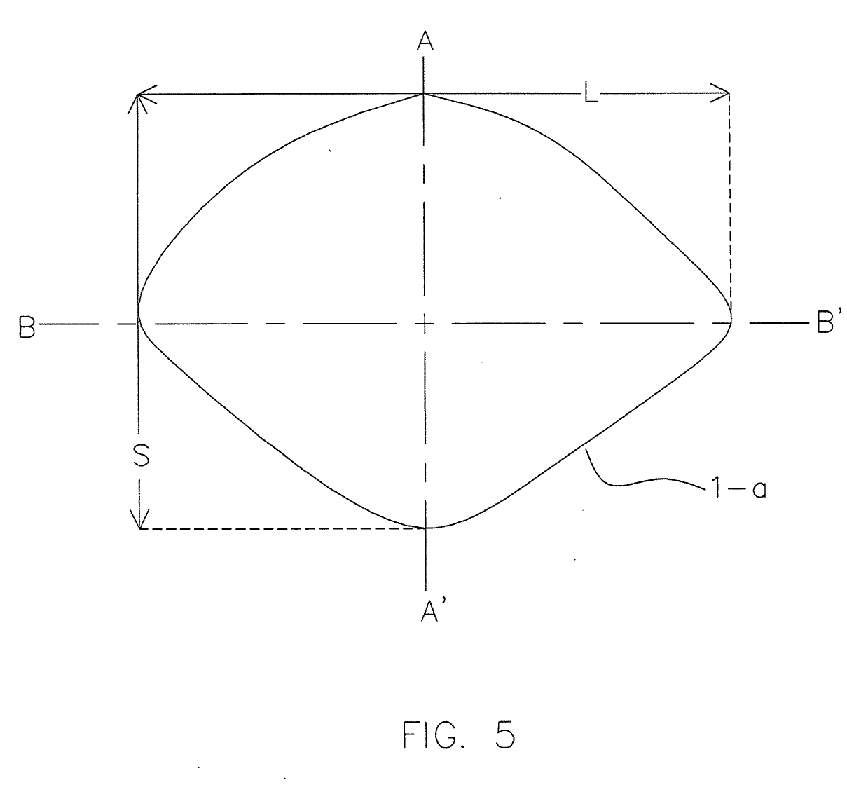

FIG. 5 is a plane view of the maxillary guidance component of the AGP of current invention.

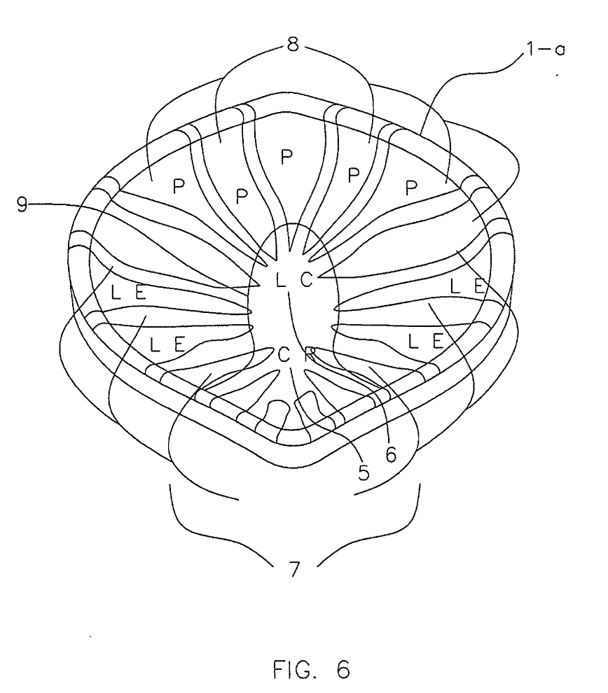

FIG. 6 is a view of the internal topography of the maxillary guidance component of the AGP showing the specific guidance of a centric relation stop, long centric area, lateral excursion guidance, and protrusive guidance.

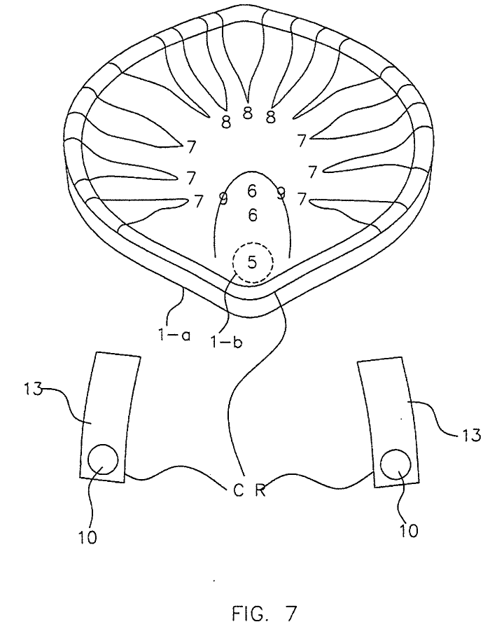

FIG. 7 is a superior transparent view of the AGP of current invention correlating the centric relation position of the TMJs coincident with the indexing of the AGP, and the available guidance to the mandibular guidance component from the maxillary guidance component from the position of centric relation.

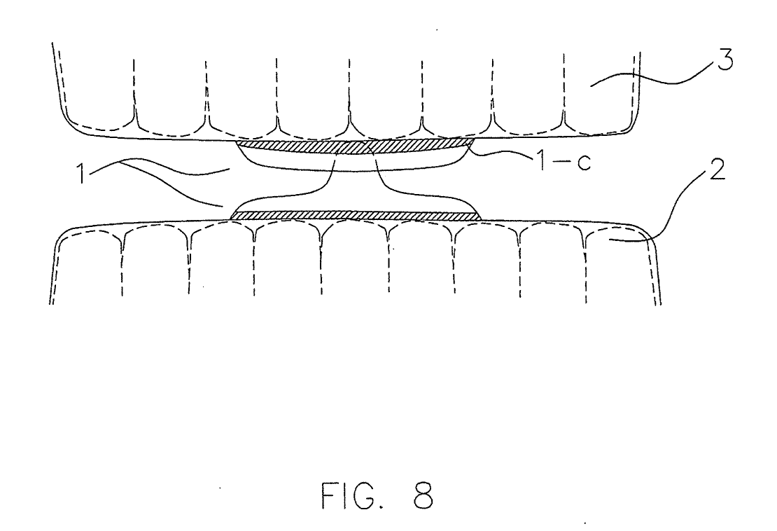

FIG. 8 is a frontal view of AGP of current invention solidly adhered on retentive pieces by adhering filler and worn by a patient.

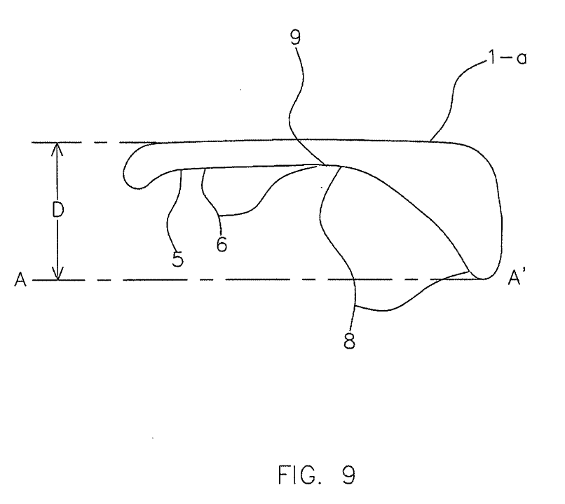

FIG. 9 is an enlarged cross-sectional view of the maxillary guidance component of the AGP according to current invention along the line A-A′.

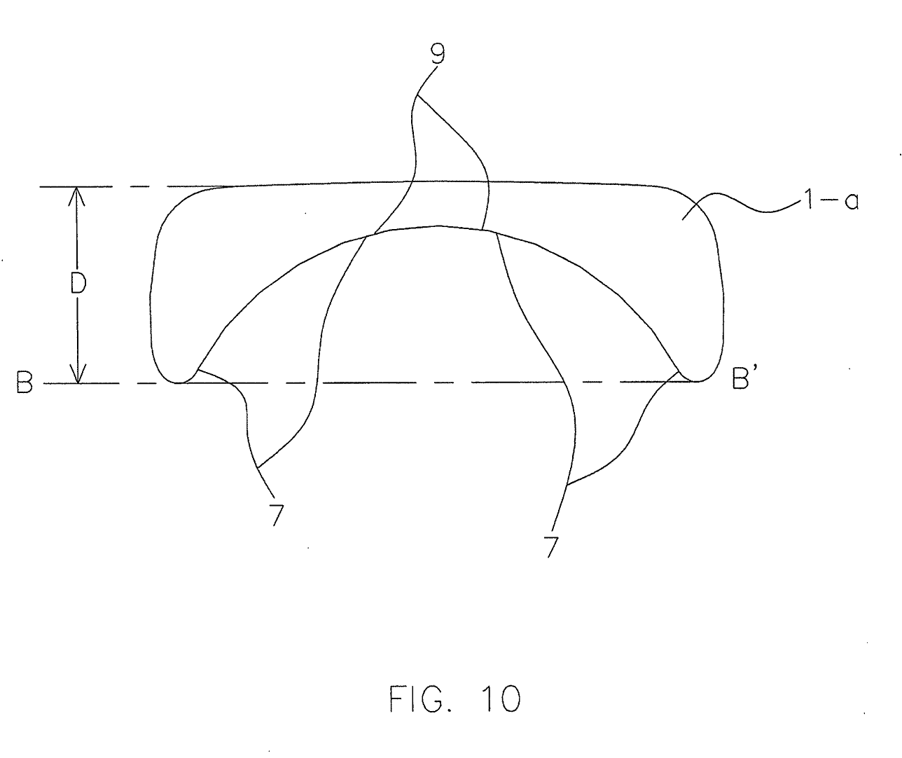

FIG. 10 is an enlarged, cross sectional view of the maxillary guidance component of the AGP according to current invention along the line B-B′.

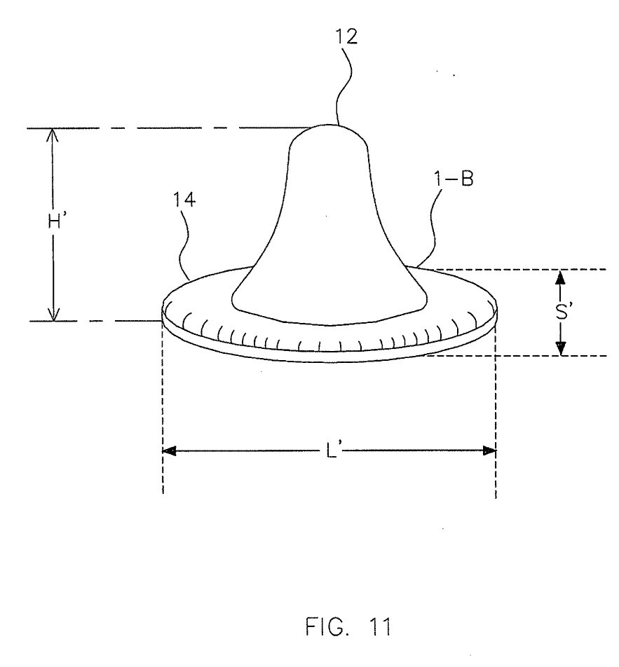

FIG. 11 is a perspective view of the mandibular guidance component of the AGP of current invention.

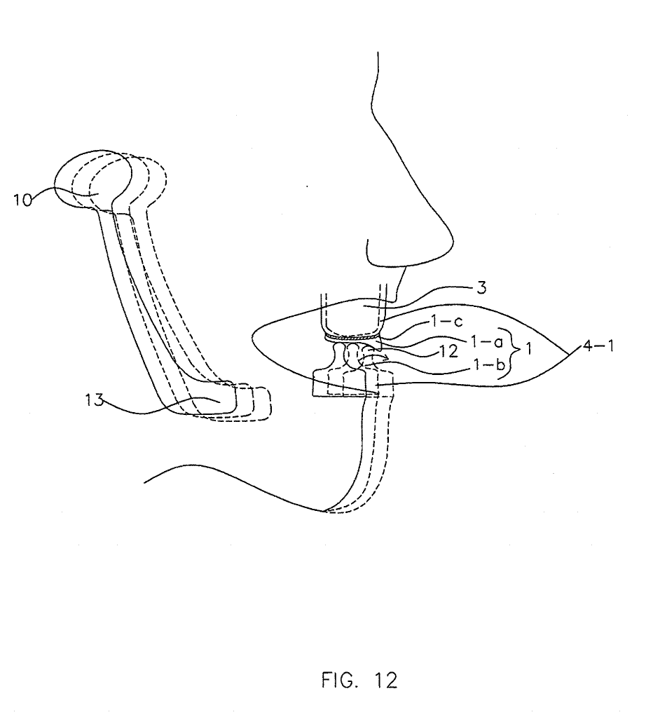

FIG. 12 is an enlarged, cross-sectional side view of movement of the mandible of a patient wearing the AGP equipped night guard.

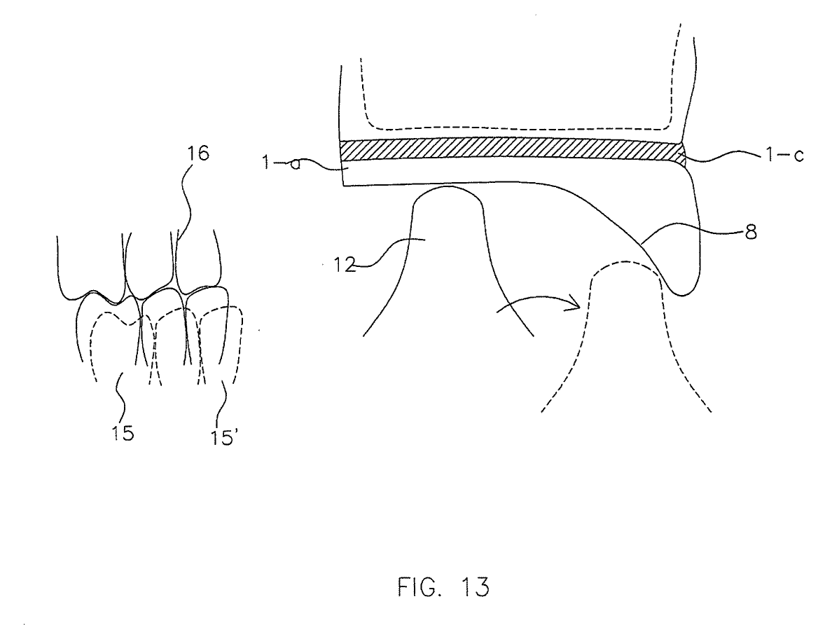

FIG. 13 is a more enlarged schematic drawing that shows how the appropriate protrusive guidance provided by the AGP of the current application eliminates posterior interferences when a patient is bruxing protrusively.

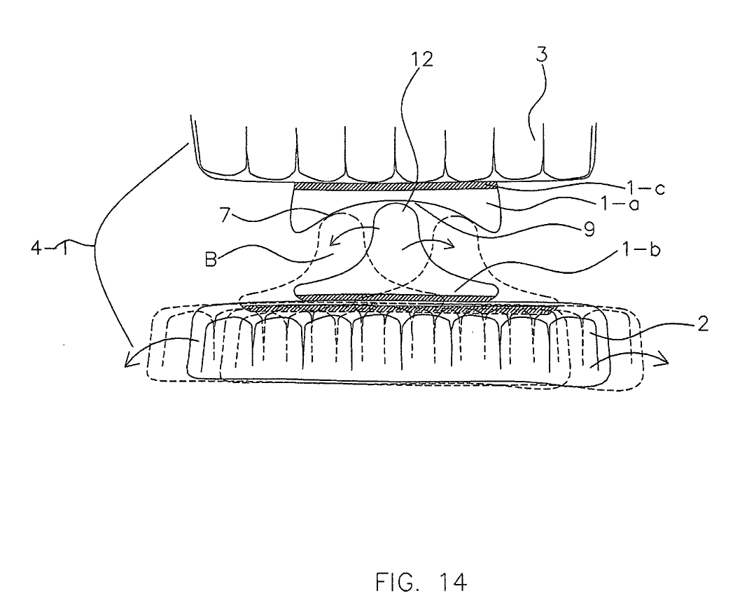

FIG. 14 is an enlarged, cross-sectional front view of movement of the mandible of a patient wearing the AGP equipped night guard.

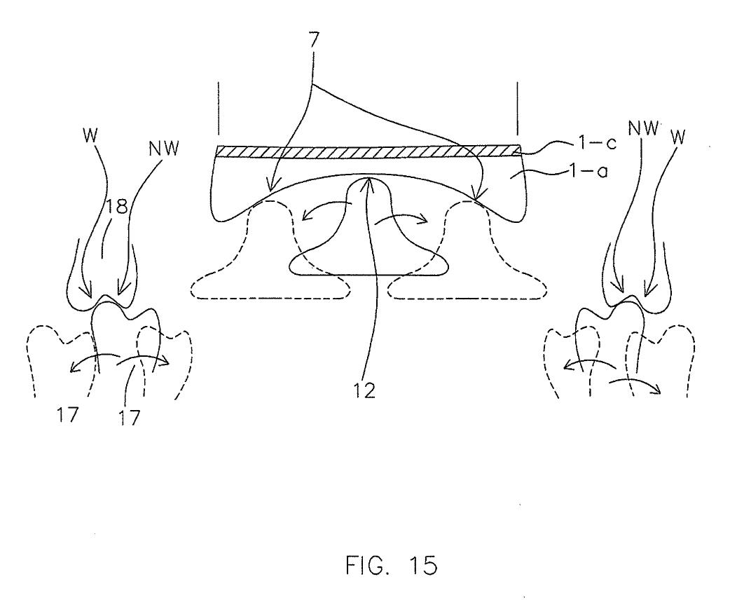

FIG. 15 is a more enlarged schematic drawing that shows the appropriate lateral guidance provided by the AGP of the current application that eliminates posterior interferences when the patient is bruxing in a left or right lateral excursion.

### DETAILED DESCRIPTION

A splint called ‘night guard’ is a hard material built on either maxillary and/or mandibular teeth. It is custom fabricated and custom adjusted by a dentist to provide anterior guidance and eliminate posterior interferences. The splint allows the patient to be free of their malocclusion and allows the patient to acquire centric relation position. Usually a dentist customizes a splint for a particular patients' malocclusion, typically an acrylic splint on one arch opposing natural teeth.

These are expensive appliances because a dentist must spend the time custom creating and custom modifying the night guard to provide anterior guidance and eliminate posterior interferences for the patient in consideration of their particular malocclusion. The patient does still inappropriately clench/brux, albeit with much less force, overall pain and damage. So, the applicant developed a night guard package that is easier to handle, more affordable for a patient and saves dentist's time.

FIG. 1 is an exploded view of Anterior Guidance Package (AGP) (1) according to current invention connected to a mandibular retention piece (2) and a maxillary retention piece (3) of a splint (4) by adhesive filler (1-c).

The Anterior Guidance Package (1) of the current invention is a pre-fabricated, pre-programmed or custom-made guidance assembly. The Anterior Guidance Package (1) of the current invention is comprised of one maxillary guidance component (1-a) and one mandibular guidance component (1-b). Those guidance components (1-a), (1-b) are attached respectively to the maxillary retention piece (3) and the mandibular retention piece (2) of a splint (4), respectively, by proper means of attachment to provide superior anterior guidance to the mandible. The proper means of attachment includes, but not limited to adhesive filler glue, screws and pins, etc. These designs can be standardized or individualized based on infinite variables and goals but generally will provide an ideal anterior guidance and the elimination of centric occlusion/centric relation discrepancies to a patient. The anterior guidance package (1) could be any of many designs.

In contrast to a splint that is customized against the dentition of the opposing dental arch or even one splint opposing another splint, the components of the Anterior Guidance Package AGP (1) according to current invention can provide a wide range of features for broad application including the replication of ideal anterior guidance of teeth as would be found in an ideal occlusion. The AGP (1) according to current invention can be of any three-dimensional patterning, steepness of inclination and many other design considerations dependent upon the purpose.

### 1. Traditional Procedure of Providing Anterior Guidance to a Patient

In order to apply an anterior guidance equipped splint to a patient in a traditional procedure the dentist would:

Create an acrylic splint on the teeth of either the maxillary or the mandibular arch. Using articulation paper to mark the contacts of the opposing teeth or an opposing splint in centric relation on the acrylic splint, the dentist will carve the acrylic developing both anterior guidance and the elimination of posterior interferences in the acrylic splint. He will polish the night guard and deliver it to the patient. This procedure must be done by a dentist who has broad knowledge of how the gnathostomatic system works. These night guards are therefore time consuming and expensive for the patient because every time the dentist creates a night guard he develops by gradual and time-consuming carving the anterior guidance and the elimination of interferences in centric relation until it fits the patient.

### 2. Procedure of Applying Pre-Programmed AGP of the Current Invention to a Patient

From the long period of practicing as a dentist the inventor found that most average adults have anterior guidance that if it were ideal, fit within specific dimensions and patterning.

FIG. 2 shows a cross-sectional view of the not as yet separated AGP (1) of current invention before it is indexed onto their respective retentive pieces. The holder (H) keeps the AGP (1) package together in its correct orientation until the components of the AGP are indexed appropriately in centric relation and in vertical dimension.

FIG. 3 shows the maxillary arch with the maxillary retentive piece (3) molded onto the maxillary teeth and the mandibular arch with a mandibular retentive piece (2) molded onto mandibular teeth hinging up in centric relation (CR).

First the operator (usually a dentist) identifies what is the first contact (FC) in centric relation (CR). When the jaw is hinged up in centric relation the position of the first contact (FC) of teeth or retentive pieces is variable dependent upon the malocclusion of that particular patient and is most often an inappropriate posterior contact.

A 1 mm sticky but removable spacer (SP) is placed on that first contact. Next the mandible is hinged again up in centric relation and the AGP (1) of current invention is indexed in the most anterior area of both the maxillary (3) and mandibular (2) retentive pieces respectively.

FIG. 4 shows the AGP (1) of current invention placed on the anterior inferior surface of the maxillary retentive piece using adhesive filler (1-c). Adhesive filler (1-c) is already placed on the surface of the AGP (1) as shown in the FIG. 2. As the jaw is hinged up in centric relation and the sticky but removable spacer (SP) touches the opposing arch at first contact (FC), the adhesive filler (1-c) is displaced from the superior and inferior surface of the AGP (1) of current invention to define the vertical dimension (VD) for a splint (4-1) that is equipped with the AGP (1) of current invention for that particular patient. Also, the maxillary guidance component (1-a) is indexed onto the maxillary retentive piece (3) and the mandibular guidance component (1-b) is indexed onto the mandibular retentive piece (2) according to the centric relation (CR) position of the mandible.

The adhesive filler (1-c) left between the retention pieces (2),(3) and guidance components (1-a),(1-b) is hardened and both components (1-a),(1-b) of the AGP(1) of current invention become rigidly affixed to their respective retentive pieces (2),(3).

Then remove the holder (H) from the AGP (1) and the mandibular guidance component (1-b) is separated from the maxillary guidance component (1-a).

The sticky but removable 1 mm spacer (SP) is removed.

The effect is that the AGP (1) of current invention is now indexed appropriately for whatever occlusion or malocclusion a patient may have to provide ideal anterior guidance and in the appropriate vertical dimension to eliminate all centric occlusion/centric relation discrepancies (or posterior interferences) after the spacer (SP) is removed in the centric relation position of that particular patient.

FIG. 5 is a plane view of the maxillary guidance component (1-a) of the AGP (1) of the current invention. The plane view of the maxillary guidance component (1-a) is a square-ovoid shape. According to dental literature and the inventors experience, it was found that the long axis (L) of the square-ovoid shape maxillary guidance component (1-a) of the AGP (1) is less than 35 mm, preferably less than 25 mm and the short axis (S) of the square-ovoid shape maxillary guidance component (1-a) is less than 20 mm, preferably 12 mm.

As shown in the FIG. 6, perspective view of the maxillary guidance component (1-a) of the AGP (1) has a flat area for a stable centric relation stop (CR) (5) extended into a further area of flat for the long centric position (LC) (6) of the mandible extending laterally and anterior into blended inclines of a concave shape for lateral excursion guidance (LE) (7) and protrusive excursion guidance (P) (8) to provide ideal anterior guidance to the patient's mandible by the mandibular guidance component (1-b) against these features of the maxillary guidance component (1-a) to minimize muscular force and avoid all posterior interferences. This feature of appropriate anterior guidance which discludes the mandible downward (inferiorly) in its excursions allows for a night guard of significantly less vertical dimension (VD) than other designs much like an ideal occlusion would.

FIG. 7 shows a superior transparent view of the AGP (1) of current invention. It shows how the AGP (1) of current invention replicates ideal anterior guidance as defined in current dental literature and the experience of the inventor. In FIG. 7 point (5) represents where the mandibular guidance component (1-b) sits at rest in the maxillary guidance component (1-a) when the condyle (10) of the temporo-mandibular joint (TMJ) (11) of the mandible is in its centric relation (CR) position. As a patient functions or bruxes his mandible, the mandibular guidance component (1-b) provides ideal anterior guidance for the mandible by means of the mandibular guidance component (1-b) functioning against the maxillary guidance component (1-a) in the position of centric relation (5), long centric (6), lateral excursions (7), and protrusive guidance (8). The AGP (1) of current invention provides ideal anterior guidance without regard to the position of teeth, the condition of teeth or missing teeth.

To apply the pre-programmed AGP (1) of the current invention to a patient the following items are needed:
1) One pre-fabricated AGP (FIG. 2 is a cross-sectional side view of the pre-fabricated AGP kit (1) before it is indexed to a patient's retentive pieces). The pre-fabricated AGP (1) kit is assembled and held by a holder (H),
2) one mandibular retention piece (2), (The mandibular retention piece could be full arch or less coverage but must be highly retentive and stiff. It would be easily moldable to the patients' lower teeth and very thin.),
3) one maxillary retention piece (3) (moldable, highly retentive, stiff and thin),
4) one 1 mm sticky but removable spacer (SP), and
5) dryer or light curing unit should be prepared.

When those items are ready:
(A) Apply to the patient the maxillary retention piece (3) and mandibular retention piece (2) on his/her teeth.
(B) Place the patient's mandible into centric relation position and identify the first contact (FC) (A point that touches first when the mandible is hinged up in centric relation. This point is highly variable from person to person dependent upon their malocclusion. It will be in the posterior segment for most people.)
(C) Then place a sticky 1 mm but removable spacer (SP) on that first contact (FC).
(D) Place the AGP (1) in the anterior inferior aspect of the maxillary retentive piece (3). The outer surface (superior and inferior) of the anterior guidance components (1-a), (1-b) is covered with adhesive filler (1-c).
(E) Hinge the mandible in centric relation up toward the maxilla and when the 1 mm sticky spacer touches (SP) the first contact (FC) index the mandibular guidance component (1-b) onto the mandibular retentive piece (2). Both components (1-a), (1-b) of the AGP(1) have now been indexed appropriately to each respective retentive piece (2),(3) in the correct vertical dimension (VD) by displacing the adhesive filler (1-c)′ to that vertical dimension. Also both components (1-a), (1-b) have been indexed appropriately anteriorly-posteriorly for that particular patients centric relation(CR) position. Both components (1-a), (1-b) of the AGP (1) should now be adhered rigidly with the 1 mm thick sticky spacer(SP) still on the first contact point.
(F) Dry or polymerize the adhesive filler (1-c) with a dryer or a light cure unit to compensate for the gap between the retention pieces (2), (3) and the anterior guidance components (1-a), (1-b) and adhere solidly and rigidly.
(G) Take the entire assembly of night guard (4-1) out of the patients' mouth. Then remove the 1 mm sticky spacer (SP) from the first contact (FC), and remove the holder (H) from the AGP (1).

The AGP (1) equipped night guard (4-1) is now ready for use.

The above described procedure provides a superior night guard to any previous method, and is much simpler to construct than any traditional or previous method of creating a night guard.

In the traditional method, carving anterior guidance and eliminating posterior interferences on an acrylic platform can take multiple appointments, and takes significant time of a dentist who has extensive knowledge of the gnathostomatic system.

But, in the new method utilizing the AGP (1), the entire process can happen in one appointment in significantly less time and could be accomplished by an individual with significantly less training.

FIG. 8 is a frontal view of the AGP (1) of the current invention solidly adhered on retention pieces (2), (3) by adhering filler (1-c) and worn by a patient.

Since the role of the anterior guidance is to limit and guide the movement of the mandible while a patient is wearing the AGP splint (4-1), a threshold (9) of continuous lateral and protrusive guidance is developed along the face of the maxillary guidance component (1-a).

FIG. 9 and FIG. 10 are enlarged cross sectional views of the maxillary guidance component (1-a) of the AGP (1) according to current invention along the lines A-A′ and B-B′ in the FIG. 5. Threshold (9) is smoothly extended to the concave internal surface of the maxillary guidance component (1-a). However, this concave internal surface may be altered to an asymmetrical concave surface based on the malocclusion or other special considerations of a particular patient. The depth (D) of the maxillary guidance component (1-a) is, including but not limited to, 1 to 5 mm, preferably 4 mm.

FIG. 11 is a perspective view of mandibular guidance component (1-b) of the AGP (1) according to current invention. The base (14) of the mandibular guidance component (1-b) has the square ovoid shape and same dimension as the maxillary guidance component (1-a) as shown in the FIG. 4.

The length of the long axis (L′) of the oval shaped mandibular guidance component (1-b) is, including but not limited to, between 15 to 35 mm. And the length of the short axis (S′) of the oval shaped mandibular guidance component (1-b) is, including but not limited to, between 8 to 20 mm.

A smooth rounded protrusion (12) is developed on one surface of the square ovoid shaped mandibular guidance component (1-b). Tip of the protrusion (12) is engaged in the flat to concave inner surface of the maxillary guidance component (1-a) and guides and limits the movement of a patients' mandible. Height of the smooth protrusion is, including but not limited to, between 1 to 6 mm, preferably 5 mm.

FIG. 12 is an enlarged, cross-sectional side view of protrusive movement of mandible/jaw (13) of a patient wearing the AGP (1) equipped night guard (4-1). When a patient, who has a habit of bruxism/clenching, wearing AGP (1) equipped night guard (4-1) of the current invention, the patient's mandible will experience appropriate protrusive guidance, guiding the jaw inferiorly.

FIG. 13 is more enlarged schematic drawing that shows how the appropriate protrusive guidance (8) provided by the AGP (1) attached to the night guard (4-1) to eliminate posterior interferences when a patient is bruxing protrusively. When a patient closes his mandible to function or brux, whether in an appropriate occlusion or a malocclusion, the intercuspation of the teeth as they mesh will bring maxillary tooth cusp inclines (16) and mandibular tooth cusp inclines (15) move into close proximity or touching (whether they have the thin retentive material on them or not). In a protrusive excursion of the mandible, without the appropriate anterior protrusive guidance of the AGP (1) of current application, these inclines (15),(16) would collide potentially damaging the teeth, but also these deviating inclines (posterior interferences) would give a proprioceptive message to the muscles to deviate over these posterior interferences, therefore stimulating the muscles to hold the mandible in this deviated position ultimately placing the TMJ into an inappropriate position and leading to muscle spasticity.

If the guidance were flat like some previous systems, the vertical dimension of the entire appliance must be increased dramatically to avoid these interferences. Therefore even when the mandible is at rest in centric relation, and not even in an excursion, the vertical dimension of the entire appliance would be so excessive, that for many patients, it could not be used or at a minimum be more uncomfortable as compared to the AGP (1) equipped night guard (4-1). The AGP (1) of current application solves the vertical dimension problem because it gives appropriate protrusive anterior guidance (8) also in a vertical way using the smooth protrusion (12) of the mandibular guidance component (1-b) guiding the mandible downward (inferiorly) therefore avoiding collisions of these tooth inclines (15), (16) or the production of unwanted and inappropriate muscle engrams, much like ideal tooth anterior guidance gives to an ideal occlusion.

FIG. 14 is an enlarged, cross-sectional front view of movement of the mandible/jaw of a patient wearing the AGP (1) equipped night guard (4-1). When a patient is experiencing bruxism/clenching and wearing the AGP (1) equipped night guard (4-1), his/her jaw/mandible (13) moves laterally left and right and receives appropriate lateral guidance, guiding the jaw downward (inferiorly).

FIG. 15 shows the appropriate lateral guidance (7) provided by the AGP (1) attached to the night guard (4-1) to eliminate posterior interferences when the patient is bruxing in a left or right lateral excursion. When a patient closes his mandible to function or brux, whether in an appropriate occlusion or a malocclusion, the intercuspation of the teeth as they mesh will bring maxillary tooth cusp inclines (18) and mandibular tooth cusp inclines (17) into close proximity or touching (whether they have the thin retentive material on them or not). In a lateral excursion of the mandible, without the appropriate anterior lateral excursion guidance of the AGP (1) of current application, these inclines (17), (18) would collide potentially damaging the teeth, but also these deviating inclines (17), (18) (posterior interferences) could produce both working (W) and non-working (NW) interferences and would give a proprioceptive message to the muscles to deviate over these posterior interferences, therefore stimulating the muscles to hold the mandible in this deviated position, ultimately placing the TMJ into an inappropriate position and leading to muscle spasticity.

If the guidance were flat like some previous systems, the vertical dimension of the entire appliance must be increased dramatically to avoid these interferences. Therefore even when the mandible is at rest in centric relation, and not even in an excursion, the vertical dimension of the entire appliance would be so excessive, that for many patients, it could not be used, or at a minimum would be more uncomfortable as compared to the AGP splint (4-1). The AGP (1) of current application solves the vertical dimension problem because it gives appropriate lateral excursion anterior guidance (7) also in a vertical way by guiding the smooth protrusion (12) of the mandibular guidance component (1-b) downward (inferiorly) much like ideal tooth anterior guidance gives to an ideal occlusion.

The completed night guard (4-1) equipped with AGP (1) of current invention provides appropriate anterior guidance for the mandible from the mandibular guidance component (1-b) which has a smooth rounded protrusion (12) closing into the maxillary guidance component (1-a) of the AGP (1) of the current invention that provides a flat area for a stable centric stop (5), an area for the mandible to assume the long centric position (6), and harmonized protrusive guidance (8) and lateral guidance (7) to the full border limits of mandibular range of motion.

The night guard (4-1) equipped with AGP (1) of current design provides ideal anterior guidance which replicates what would be found in an ideal occlusion.

The ideal anterior guidance provided by the night guard (4-1) equipped with the AGP (1) significantly reduces the muscle forces of bruxism because of the mechanical advantageous position of being anterior to the muscle power used to close the mandible. The night guard (4-1) equipped with AGP (1) of current invention provides a stable centric stop (5) and appropriate anterior guidance of long centric position (6), lateral excursion guidance (7), harmonized with protrusive guidance (8), and threshold (9) to avoid/eliminate all centric occlusion/centric relation discrepancies (posterior interferences), with a minimal vertical dimension increase, therefore allowing the condyles to seat into their most comfortable and best stress bearing positions at a reasonable vertical dimension.

The night guard (4-1) equipped with the AGP (1) of current invention eliminates deviating tooth contacts with minimal vertical dimension increase. By eliminating the collision of deviating inclines of teeth in a malocclusion there will be no proprioceptive message to muscles to deviate around that interference. When the muscle stops being stimulated into holding the mandible in a deviated position, then normal muscle activity can resume and spasticity will cease. And finally, the AGP (1) incorporated onto an AGP splint (4-1) will accomplish all this without regard to missing teeth, weakened teeth or the malocclusion of teeth because teeth are not used for guidance in any way and all interferences are eliminated.

The pre-fabricated AGP (1) of the current invention can be made in many different shapes based on the malocclusion or particular problem of the patient, such as a TMJ internal derangement. For example, one could construct an AGP with asymmetrical lateral guidance (7) of the maxillary guidance component (1-a).

The pre-fabricated AGP (1) of the current application can be provided combined with specialized retentive pieces, which will be disclosed in another application of the inventor.

The AGP (1) of the current application can be easily fabricated with the aid of CAD-CAM technology with specific specifications for a particular patient considering their specific malocclusion, which will be disclosed in another application of the inventor.

The AGP (1) of the current application can be specifically produced that can move the TMJ off centric relation or in non-traditional pathways for the treatment of specific TMJ treatment and other maladies, which will be disclosed in another application of the inventor.

The AGP (1) of the current application can be utilized with alternative ways of determining what position other than centric relation position that the mandible should rest and be guided, which will be disclosed in another application of the inventor.

What is claimed is:

1. A pre-fabricated anterior guidance package (AGP) comprising:
a maxillary guidance component configured to be rigidly adhered to a maxillary retentive piece configured to be placed about a maxilla of a user, the maxillary guidance component having a downwardly-facing concave bottom surface;
a mandibular guidance component configured to be rigidly adhered to a mandibular retentive piece configured to be placed about a mandible of the user, the mandibular guidance component having a smooth protrusion that forms a convex tip, the convex tip being configured to engage the concave bottom surface of the maxillary guidance component to place the user's maxilla and mandible in a centric relation position; and
a removable holder simultaneously attached to the mandibular and maxillary guidance components and maintaining a correct orientation of the mandibular guidance component relative to the maxillary guidance component until the mandibular and maxillary guidance components are rigidly adhered, in the correct orientation, to the mandibular and maxillary retentive pieces, respectively,
wherein the correct orientation of the mandibular guidance component relative to the maxillary guidance component is such that anterior guidance may be provided to the mandible of the user from the mandibular guidance component functioning against the maxillary guidance component when the mandibular and maxillary guidance components are rigidly adhered, in the correct orientation, to the mandibular and maxillary retentive pieces, respectively,
wherein the maxillary guidance component is configured to, when the convex tip of the mandibular guidance component is engaged with the concave bottom surface of the maxillary guidance component in the correct orientation, downwardly direct the convex tip of the smooth protrusion of the mandibular guidance component in response to lateral movement of the mandibular guidance component relative to the maxillary guidance component.

2. The pre-fabricated AGP of claim 1, wherein the maxillary guidance component has a long axis with a length between 15 mm and 35 mm and a short axis length between 8 mm to 15 mm.

3. The pre-fabricated AGP of claim 1, wherein the mandibular guidance component has a long axis length between 15 mm and 35 mm and a short axis length between 8 mm to 15 mm.

4. The pre-fabricated AGP of claim 1, wherein the convex tip of the smooth protrusion of the mandibular guidance component extends between 1 mm and 6 mm from a base of the mandibular guidance component.

5. The pre-fabricated AGP of claim 1, wherein the maxillary guidance component is rigidly adhered to a maxillary retentive piece and the mandibular guidance component is rigidly adhered to a mandibular retentive piece by adhesive filler.

6. The pre-fabricated AGP of claim 1, wherein the mandibular and maxillary retentive pieces are pre-manufactured.

7. The pre-fabricated AGP of claim 1, wherein
the maxillary guidance component is separated from the maxillary retention piece,
the mandibular guidance component is separated from the mandibular retention piece, and the removable holder maintains the correct orientation of the mandibular guidance component relative to the maxillary guidance component.

8. A pre-fabricated dental guidance device comprising:
a mandibular retentive piece for placement about a mandible of a user;
a mandibular guidance component attachable to the mandibular retentive piece, the mandibular guidance component having a convex top edge;
a maxillary retentive piece for placement about a maxilla of the user;
a maxillary guidance component attachable to the maxillary retentive piece, the maxillary guidance component having a concave bottom surface configured to receive the convex top edge of the mandibular guidance component; and
a removable holder simultaneously attached to the mandibular and maxillary guidance components and maintaining a correct orientation of the mandibular guidance component relative to the maxillary guidance component until the mandibular and maxillary guidance components are rigidly adhered, in the correct orientation, to the mandibular and maxillary retentive pieces, respectively,
wherein the correct orientation of the mandibular guidance component relative to the maxillary guidance component is such that anterior guidance may be provided to the mandible of the user from the mandibular guidance component functioning against the maxillary guidance component when the mandibular and maxillary guidance components are rigidly adhered, in the correct orientation, to the mandibular and maxillary retentive pieces, respectively.

9. The pre-fabricated dental guidance device of claim 8, wherein the maxillary retentive piece is pre-manufactured, and the maxillary guidance component is rigidly affixed to the pre-manufactured maxillary retentive piece using adhesive filler.

10. The pre-fabricated dental guidance device of claim 8, wherein the mandibular retentive piece is pre-manufactured, and the mandibular guidance component is rigidly affixed to the pre-manufactured mandibular retentive piece using adhesive filler.

11. The pre-fabricated dental guidance device of claim 8, wherein the concave bottom surface of the maxillary guidance component comprises a front protrusion and a rear protrusion, the front protrusion downwardly extending further than the rear protrusion.

12. The pre-fabricated dental guidance device of claim 11, wherein the removable holder is configured to maintain the correct orientation of the mandibular guidance component relative to the maxillary guidance component such that the convex top edge of the mandibular guidance component abuts the concave bottom surface of the maxillary guidance component between the front and rear protrusions.

13. The pre-fabricated dental guidance device of claim 11, wherein the concave bottom surface of the maxillary guidance component is downwardly-facing and further comprises a left protrusion and a right protrusion, the left and right protrusions downwardly extending the same distance.

14. A pre-fabricated anterior guidance package (AGP) comprising:
a mandibular guidance component capable of being attached to a pre-manufactured maxillary retentive piece configured to be placed about a mandible of a user, the mandibular guidance component having a convex top edge;
a maxillary guidance component capable of being attached to a pre-manufactured mandibular retentive piece configured to be placed about a maxilla of the user, the maxillary guidance component having a downwardly-facing concave bottom surface configured to receive the convex top edge of the mandibular guidance component; and
a holder simultaneously attached to the mandibular and maxillary guidance components and configured to maintain a pre-programmed orientation of the mandibular guidance component relative to the maxillary guidance component until the mandibular and maxillary guidance components are attached, in the pre-programmed orientation, to the pre-manufactured mandibular and maxillary retentive pieces, respectively.

15. The pre-fabricated anterior guidance package (AGP) of claim 14, wherein the holder is removable.

16. The pre-fabricated anterior guidance package (AGP) of claim 15, wherein the maxillary guidance component is adhered to the pre-manufactured maxillary retentive piece by adhesive filler.

17. The pre-fabricated anterior guidance package (AGP) of claim 15, wherein the mandibular guidance component is adhered to the pre-manufactured mandibular retentive piece by adhesive filler.

18. The pre-fabricated anterior guidance package (AGP) of claim 15, wherein the concave bottom surface of the maxillary guidance component comprises a front protrusion and a rear protrusion, the front protrusion downwardly extending further than the rear protrusion.

19. The pre-fabricated anterior guidance package (AGP) of claim 18, wherein the removable holder is simultaneously attached to the mandibular and maxillary guidance components and configured to maintain the pre-programmed orientation of the mandibular guidance component relative to the maxillary guidance component such that the convex top edge of the mandibular guidance component abuts the concave bottom surface of the maxillary guidance component between the front and rear protrusions.

20. The pre-fabricated dental guidance device of claim 18, wherein the concave bottom surface of the maxillary guidance component is downwardly-facing and further comprises a left protrusion and a right protrusion, the left and right protrusions downwardly extending the same distance.
